(19) United States
(12) Patent Application Publication
(10) Pub. No.: US 2014/0175314 A1
(43) Pub. Date: Jun. 26, 2014

(54) STRAIN RELIEVING VALVE ASSEMBLY WITH FLOW BLOCKING

(71) Applicants: Louise Jul Bondo, Copenhagen 0 (DK); Karsten Videbæk, Jyllinge (DK)

(72) Inventors: Louise Jul Bondo, Copenhagen 0 (DK); Karsten Videbæk, Jyllinge (DK)

(73) Assignee: IV Valve ApS, Copenhagen 0 (DK)

(21) Appl. No.: 14/115,889

(22) PCT Filed: May 3, 2012
(86) PCT No.: PCT/EP2012/058147

§ 371 Date: Feb. 7, 2014

(30) Foreign Application Priority Data
May 6, 2011 (DK) PA 2011 70223

(51) Int. Cl. A61M 39/26; F16L 29/02

(52) U.S. Cl. CPC A61M 39/26; F16L 29/02
USPC 251/149.6

(57) ABSTRACT

The present invention relates to a valve assembly for insertion in a liquid flow path between a catheter or drain and a liquid bag or container. The valve assembly comprises a first valve member detachably coupled to a second valve member or coupling member by mating locking members. A first controllable valve is adapted to selectively block or enable liquid flow through the first valve member in accordance with a valve control mechanism which is responsive to a coupling state of the first and second locking members.

The present invention relates to a valve assembly for insertion in a liquid flow path between a catheter or drain and a liquid bag or container. The valve assembly comprises a first valve member detachably coupled to a second valve member or coupling member by mating locking members. A first controllable valve is adapted to selectively block or enable liquid flow through the first valve member in accordance with a valve control mechanism which is responsive to a coupling state of the first and second locking members.

### BACKGROUND OF THE INVENTION

Indications for establishing intravenous access to a patient's vein using a peripheral vein catheter or IV-catheter may be a need for administration of liquids, fluids, drugs or nutrients or transfusion of blood or blood products. In a peripheral vein catheter a small, flexible tube is used to deliver fluids into the body. Conventional peripheral vein catheters, which have been used for decades, for example a Venflon™, often comprise a flexible tube and a trocar at one end for puncture of the patient's vein. The peripheral vein catheter may comprise a hollow main body with a female termination plug. To establish liquid connection to the peripheral vein catheter, a delivery tube, for example comprising a mating male plug, is attached or coupled to the female plug in a sealingly manner. The delivery tube leads to a container, bag or vessel holding the appropriate type of liquid drugs, nutrients etc.

The peripheral vein catheter must be fastened or secured to the patient's skin at the entry site or puncture site of a catheter needle for example with non-allergic tape, bandage, patches or an elastomeric glove etc. This is necessary because the vein itself offers little or no resistance to movement of the catheter tube and even the weighting of the delivery tube may be enough to pull out the catheter unless it is safely anchored to the patient's body. Consequently, it is of outmost importance to safely secure position of the peripheral vein catheter to avoid displacement during drug administration. However, despite attempts to secure the catheter as described above, the catheter is often vulnerable to dislodging forces, for example caused by deliberate or accidental patient movement, that may displace the catheter tube from its proper position inside the patient's vein or even entirely dislodge the catheter tube from the patient's vein. Another consequence of such movement may be detachment of a liquid connection between the delivery tube and a liquid inlet port of the peripheral vein catheter. This kind of accidental dislodging or detachment of the catheter (or drain for that matter) may be caused by patients that are confuse, heavily medicated or sleepy etc. and tend to forget the presence of the peripheral vein catheter and its associated delivery tubing when moving around. If the coupling between the delivery tube and a liquid inlet port is broken, liquid drug is able to flow freely out of the outlet port of the delivery tube and the patient's blood can flow freely out of the liquid inlet port of the peripheral vein catheter. This situation leads to waste of liquid and medication and blood loss for the patient. The accidental outpour of liquid and medicine is furthermore often supported in an undesirable manner by an elevated placement of the liquid container, bag or vessel on an IV-pole or on a pole connected to the patient's bed.

On the other hand, if the IV-catheter is entirely dislodged from the patient's vein, this situation requires unnecessary insertion of a new IV-catheter at the expense of unnecessary pain to the patient. It also wastes valuable time for the IV-therapist (doctor or nurse) and imparts unnecessary costs for a hospital. The patient is also subjected to an increased risk of skin exit-site infection or catheter-related bloodstream infection and the opportunities for inserting a new IV-catheter are reduced because of limited insertion sites (limited vessel). Furthermore, even if the dislodging forces fail to entirely dislodge the peripheral vein catheter, numerous types of damage may nevertheless be imparted such as shifting the IV-catheter to a subcutaneous position where it may cause edema, Phlebitis or infections.

Consequently, there exists a need for methods and devices that can limit dislodging forces applied to in-situ positioned catheters and drains, such as peripheral vein catheters, to allow the catheter or drain to stay in place at the puncture site during patient movements. Furthermore, a mechanism and devices that can automatically stop the leakage of blood from the patient and leakage of liquids from the liquid container or bag is highly desirable when the liquid coupling between the peripheral vein catheter and the delivery tube is broken accidentally.

### SUMMARY OF THE INVENTION

A first aspect of the invention relates to a valve assembly for insertion in a liquid flow path between a drain or catheter, such as a peripheral vein catheter, and a liquid vessel or container. The valve assembly comprises a first valve member comprising:

an inlet port connectable to a first delivery tube and arranged at a first side of the first valve member,

a first coupling port arranged at a second side of the first valve member,

a first locking member,

a first controllable valve adapted to selectively block or enable liquid flow between the inlet port and the first coupling port in accordance with a valve control mechanism. The valve assembly further comprises a coupling member comprising: an outlet port connectable to a second delivery tube and arranged at a first side of the coupling member,

a second coupling port arranged at a second side of the coupling member,

a second locking member configured for mating engagement with the first locking member on the first valve member. The valve control mechanism is responsive to a coupling state of the first and second locking members.

In accordance with the invention, the valve control mechanism may be adapted to solely block liquid flow through the first valve member, i.e. between the inlet port and the first coupling port. The first valve member is preferably fluidly coupled to the liquid vessel or container through the inlet port such that spill or outpour of costly drugs from the liquid vessel is prevented by an automatic blocking operation of the first controllable valve in case the first valve member and the coupling member are accidentally dislodged or disconnected. In addition to costs savings, the automatic blocking of liquid flow through the first valve member also minimizes uncertainty regarding the actual drug dose delivered to the patient if the valve assembly is accidentally disconnected during drug or liquid administration. This is highly beneficial for drug administration safety. In a preferred embodiment, the coupling member comprises a coupling mechanism that is shaped and sized to fit a mating or cooperating coupling mechanism of the drain or the catheter. In this embodiment, the skilled person will appreciate that the coupling member may be integrally formed with the drain or the catheter. In the latter embodiment, the number of separate components or items of the complete liquid flow path can be minimized.

According to a preferred embodiment of the invention, the coupling member comprises a second controllable valve for selectively blocking or enabling liquid flow between the outlet port and the second coupling port of the coupling member in accordance with the valve control mechanism.

The presence of the first and second controllable valves under control of the valve control mechanism can ensure automatic blocking of the liquid flow paths between:
1) The valve assembly and the catheter or drain; and
2) The valve assembly and the liquid container or bag,
   in response to detachment or disconnection of the first and second valve members during administration of liquids or drugs from the liquid container through the catheter, e.g. comprising a Venflon, to the patient. Likewise, during drainage or outflow of bodily fluids from the patient through the drain, the presence of the first, and optionally the second, controllable valve may at least block the outflow of bodily fluids from the patient and preferably also any outflow of already collected bodily fluids in the liquid container. The disconnection may for example be caused by deliberate or accidental movement of the patient's arm or body as discussed above. Consequently, the spill of costly drugs from the liquid vessel and outflow of blood or liquids from the patient's body can both be prevented by the blocking actions of the first and second controllable valve members.

The inlet port may be attached or fastened to the first delivery tube in a permanent manner for example by gluing, bonding or welding. The outlet port of the coupling member may be bonded or fastened to the second delivery tube in a permanent manner by the same methodology to provide an integral tube assembly as explained below in further detail. In another embodiment, the outlet port of the coupling member may be attached, preferably fixedly, directly to a mating coupling port and locking of the catheter or drain so as to provide a catheter or drain wherein the second delivery tube is integrated. This embodiment may accordingly provide an integral valve and valve control mechanism in the catheter or drain. Each of the first and second valve members preferably comprises two or more separate components such as a hollow housing enclosing a separate valve member. The first and second coupling ports may be provided in respective ones of the separate valve members. The first and second valve members are preferably fabricated as injection moulded thermoplastic elements. Each of the first and second coupling ports may comprise a single aperture or a plurality of apertures or openings. The inlet port, the first coupling port, the second coupling and the outlet port are preferably aligned about a longitudinal axis of the valve assembly.

In a preferred embodiment of the invention, the first and second locking members are configured to transit from an engaged coupling state to a detached coupling state at a well-defined predetermined detachment force. This predetermined detachment force may vary according to requirements of a particular application. Practical experiments conducted by the inventor suggest that the predetermined detachment force may lie between 0.5 N and 20 N, preferably between 1 N and 10 N, measured in a longitudinal axial direction of the valve assembly. This enables the coupling between the first and second valve members to break before the IV-catheter is dislodged or displaced from/in the patient's body. In one embodiment, the first and second locking members comprise a mating annular ridge and groove structure. The predetermined detachment force can be controlled by adjusting radial dimensions of the mating ridge and groove.

The first and second locking members are preferably configured to provide a user adjustable detachment or retention force for example adjustable by a medical professional in accordance with yet another advantageous embodiment of the invention. This allows a nurse, doctor or similar medical professional to conveniently adapt the retention force to specific needs of a particular clinical situation such as the abilities of a patient and the strength of the fixation of the peripheral vein catheter on the patient's body. From a manufacturing point of view, the adjustable retention force allows a significant reduction in the required number of separate variants of the present valve assembly to cover a certain range of applications without compromising individual adaptation to a particular clinical situation. In one such embodiment, an axially displaceable third locking member is surrounding and engaging at least one of the first and second locking members to provide a user adjustable detachment or retention force. The third locking member is preferably rotatably mounted about one of the first and second locking members such that rotation of the third locking member leads to axial displacement of the third locking member relative to the first and/or second locking member. To support the rotatable mounting of the third locking member, an interior surface of the third locking member and an outer surface of the first or the second locking member may comprise mating threads.

The rotatable third locking member preferably comprises a corrugated outer surface or other type of frictionally enhanced outer surface to facilitate grip and manipulation by the medical professional.

The first controllable valve may comprise a first hollow hub having formed therein a plurality of sideward facing openings and a first axially displaceable hollow member surrounding at least a portion of the first hollow hub to selectively open or block the plurality of sideward facing openings. The second controllable valve may likewise comprises a second hollow hub having formed therein a plurality of sideward facing openings and a second axially displaceable hollow member surrounding at least a portion the second hollow hub to selectively open or block the plurality of sideward facing openings. In this context, the term “sideward facing openings” is to be understood as openings steering liquid outlet or expel in a direction substantially perpendicular to the longitudinal axis of the valve assembly. In this embodiment, the first and second valve members may further be configured to move each of the first and second axially displaceable hollow members between a proximal position, where the respective pluralities of sideward facing openings are unblocked or open, and a distal position, where the respective pluralities of sideward facing openings are blocked by the first and second axially displaceable hollow members. Each of the first and second axially displaceable hollow members may comprise an annular inner surface contour mating to, and slidingly engaging, a corresponding cylindrical outer contour of the hollow hubs.

Each of the first and second axially displaceable hollow members may be displaced from the proximal to the distal position by a spring force. In one such embodiment, the first axially displaceable hollow member comprises a first spring configured to engage a circumferential collar on the first hollow hub; and the second axially displaceable hollow member comprises a second spring configured to engage a circumferential collar on the second hollow hub. The first and second springs may be manually compressed during attachment of the first and second valve members such that the respective pluralities of sideward facing openings are exposed due to the proximal arrangement of the first and second axially displaceable hollow members. Upon detachment of the first and second valve members, the respective spring forces supplied by the compressed first and second springs may displace respective ones of the first and second axially displaceable hollow members to their distal positions where the respective pluralities of sideward facing openings are blocked.

As mentioned above, the first and second axially displaceable hollow members may be adapted for sliding engagement with an outer contour of the respective hollow hubs. In one embodiment, the first hollow hub and the first axially displaceable hollow member are arranged in sliding engagement and co-axially about the longitudinal axis of the valve assembly. The second hollow hub and the second axially displaceable hollow member are likewise arranged in sliding engagement and coaxially about the longitudinal axis of the valve assembly.

As mentioned above, the first and second springs may be compressed during assembly of the first and second valve members in connection with displacing the first and second axially displaceable hollow members towards their proximal positions so as to open the respective pluralities of sideward facing openings for liquid flow. According to an embodiment, the first valve member comprises a first hollow housing at least partly surrounding the first hollow hub and the second valve member comprises a second hollow housing at least partly surrounding the second hollow hub. The first and second hollow housings are configured to engage and axially displace the first and second hollow hubs, respectively, to axially compress the first and second springs against their respective circumferential collars.

A travel or displacement distance of each of the first and second axially displaceable hollow members relative to the first and second hollow hubs, respectively, from the proximal position to the distal position preferably lies between 1 mm and 10 mm. In a preferred embodiment of the invention, the first and second controllable valves comprise a compressible elastomeric member. According to this embodiment, the first controllable valve comprises a first compressible elastomeric member having the first coupling port formed therein. In addition, the second controllable valve comprises a second compressible elastomeric member having the second coupling port formed therein. The valve control mechanism is configured to compress the first and second compressible elastomeric members to change shapes of the first and second coupling ports. The first and second coupling ports may be closed to block liquid flow from the inlet port to the first coupling port and block liquid flow from the second coupling port to the outlet port in an uncompressed or unbiased state of the elastomeric member. The first and second coupling ports may be opened in the compressed or biased state of the respective elastomeric members to enable liquid flow. According to one such embodiment, the first coupling port comprises a first membrane having a substantially straight slit formed therein and the second coupling port comprises a second membrane having a substantially straight slit formed therein. The valve control mechanism may be configured to change the first coupling port between an expanded state, enabling liquid flow, and a collapsed state, blocking liquid flow. The valve control mechanism is preferably also configured to change the second coupling port between an expanded state, enabling liquid flow, and a collapsed state, blocking liquid flow. In this embodiment, the substantially straight slits are expanded to respective openings in the first and second membranes. The first and second membranes are configured to block liquid flow through the first and second valve members, respectively.

In a preferred embodiment, the first and second compressible elastomeric members are integrally formed as a single elastomeric element, for example an injection moulded element, to minimize component count and assembly costs.

The first and second locking members may be designed in variety of ways using cooperating or mating mechanical features of the first and second valve members to fasten the first and second valve members in the attached coupling state. In one embodiment, the first locking member comprises a circumferential ridge or collar arranged on the first valve member and the second locking member comprises a mating circumferential groove or indentation arranged on the second valve member. The first locking member, such as the circumferential ridge, is preferably formed integrally with the first valve member but may alternatively be formed as a separate member that is attached to the first valve member by adhesive agents, bonding, press-fitting etc. The same applies for the second locking member.

By varying appropriate dimensions of the circumferential ridge and the mating circumferential groove, the predetermined detachment force may be adjusted. Likewise, the predetermined detachment force may be adjusted by varying material properties of the first and/or second valve member(s). Naturally, if the first and second locking members are configured to provide the user adjustable detachment force mentioned above, the range of the detachment force may be adjusted by varying the appropriate dimensions and/or material properties.

Dimensions of the valve assembly may vary according to particular medical applications. In a set of preferred embodiments of the invention, a length of the valve assembly lies between 20 mm and 40 mm in an engaged coupling state of the first and second valve members. The valve assembly preferably has a maximum cross-sectional dimension between 8 mm and 15 mm in an engaged coupling state of the first and second valve members. These dimensions are often suitable to interface to normal dimensions, often inner diameters between 3 mm and 8 mm, of the first and second delivery tubes at the inlet port and outlet port, respectively.

In another embodiment, the valve assembly comprises a visible colour indicator responsive to the coupling state of the first and second locking members such that the visible colour indicator has a first colour when the first and second valve members are in the attached coupling state and a different, second, colour when the first and second valve members are in the detached coupling state. The first colour may for example be green and the second colour red to signal an enabled and disrupted liquid flow path, respectively, to the medical professional. In yet another embodiment of the present invention which comprises the previously discussed adjustable detachment force, the valve assembly comprises a window with a visible scale that indicates a current magnitude of the detachment force. This may be window displaying a threaded section of a cylindrical housing member.

In yet another embodiment of the invention, the first valve member and the coupling member are rotatable relative to each other about a longitudinal axial direction of the valve assembly in an engaged coupling state of the first and second locking members. The first valve member preferably comprises a cylindrical housing enclosing a co-axially arranged mating cylindrical valve mechanism. The coupling member preferably comprises a cylindrical hollow housing. The rotatable feature prevents troublesome buckling or bending of an attached elastomeric delivery tube coupled to the valve assembly. This kind of buckling tends to be induced by patient movement and can often create undesired dislodging forces to the catheter in the patient's body during drug administration.

Another aspect of the invention relates to a tube assembly for establishing liquid coupling between a distant port and a proximal port of a delivery tube e.g. an IV-line. The tube assembly comprises a valve assembly according to any of the above-described embodiments disposed in-between the distant port and the proximal port such that the inlet port is coupled to a first intermediate port of the delivery tube and the outlet port coupled to a second intermediate port of the delivery tube. The inlet port of the valve assembly and the first intermediate port of the delivery tube may be integrally formed. Likewise, the outlet port and the second intermediate port of the delivery tube may be integrally formed in which case the tube assembly has only one single detachment location, i.e. between the first and second valve members. Furthermore, the detachment force required to disconnect the tube assembly at valve assembly may be well-defined and lie between 0.5 N and 20 N. The distant port of the delivery tube may be coupled to a liquid container or bag holding medicine or water etc. for administration to a patient through the catheter or the distant port may be coupled to a bag or container collecting bodily fluids expelled from the patient through the drain. The proximal port of a delivery tube is intended for coupling to a drain, tube or catheter inserted in the patient's body, for example in a vein or subcutaneously. The present tube assembly may accordingly be formed as a single item replacing a conventional type of flexible medical delivery tube but with the ability to automatically break the liquid connection between the liquid container and the drain or catheter if too large stress forces are imparted to the tube assembly, i.e. stress forces exceeding the predetermined detachment force of the valve assembly. This property prevents accidental dislodging of the drain or catheter and vein damage as previously explained. In addition, the liquid flow out of one, or preferably both, of the released ends of the IV-line, i.e. at the first and second coupling ports, is automatically blocked by the operation of the first and second controllable vents.

### BRIEF DESCRIPTION OF THE DRAWINGS

A preferred embodiment of the invention will be described in more detail in connection with the appended drawings, in which:

FIG. 1 illustrates a valve assembly in accordance with a first embodiment of the invention in an exploded perspective view,

FIG. 2 is a perspective view of the valve assembly in an engaged or attached state,

FIG. 3A is a longitudinal cross-sectional view of the valve assembly in a disengaged or detached state,

FIG. 3B is a longitudinal cross-sectional view of the valve assembly in an engaged or attached state,

FIG. 4A is a longitudinal cross-sectional view of the second embodiment of the valve assembly in a disengaged or detached state,

FIG. 4B is a longitudinal cross-sectional view of a second embodiment of the valve assembly in an engaged or attached state,

FIG. 5 is an exploded perspective view a valve assembly in accordance with a third embodiment of the invention,

FIG. 6 is a perspective view of the valve assembly in an engaged or attached state in accordance with the third embodiment of the invention,

FIG. 7 is a longitudinal cross-sectional view of the valve assembly in accordance with the third embodiment in the engaged or attached state,

FIG. 8A is a transverse cross-sectional view of the valve assembly in accordance with the third embodiment in a disengaged or detached state; and

FIG. 8B is a transverse cross-sectional view of the valve assembly in accordance with the third embodiment in the engaged or attached state.

### DETAILED DESCRIPTION OF PREFERRED EMBODIMENTS

The embodiments of the present the valve assembly that are described in detail below are specifically adapted for use with IV-catheters. However, the skilled person will understand that detachable valve assemblies in accordance with the present invention are highly useful for other types of catheter/tubes inserted into a duct, vessel or other parts of the patient's body cavity. These other types of catheters may for example comprise urinary catheters, epidural catheter or chest tubes for drainage of pleural effusion, peritoneal catheters for drainage of ascites, central venous catheters and ordinary medical delivery tubes.

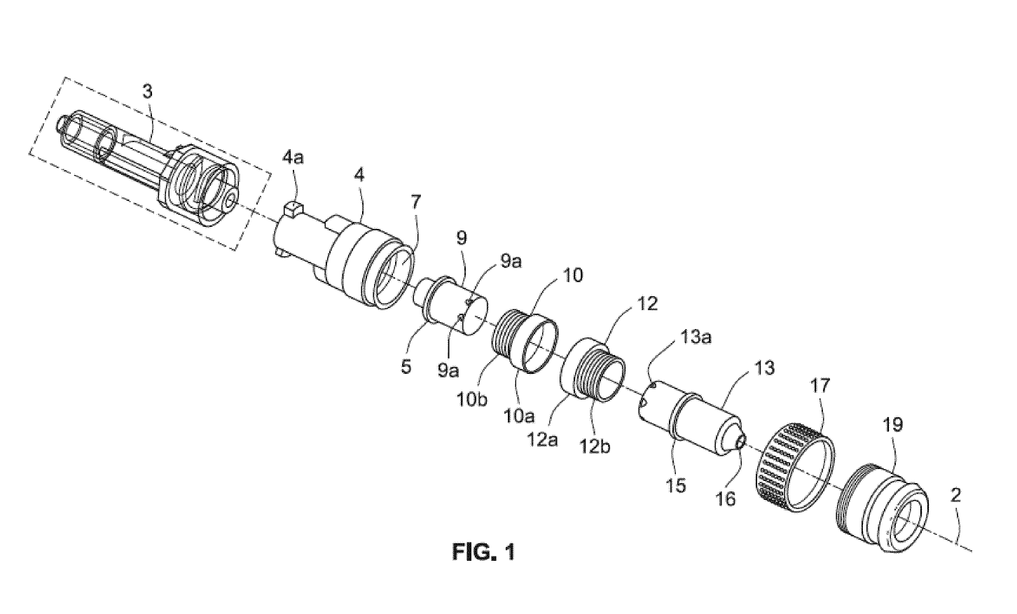

FIG. 1 illustrates a valve assembly 1 in accordance with a first embodiment of the invention in an exploded perspective view. An adapter 3 does not form part of the present valve assembly 1 but may be integrated with a Venflon type of catheter (not shown) intended for coupling to a male socket arranged on the present valve assembly 1.

The valve assembly 1 comprises a first valve member or assembly comprising a first cylindrical housing member 4, a first hollow hub 9 and a first axially displaceable hollow member 10 or first hollow member 10. The valve assembly 1 comprises further a second valve member or assembly comprising a second cylindrical housing member 19, a second hollow hub 13 and a second axially displaceable hollow member 12. An inlet port (not shown) is formed in a distal end of the first cylindrical housing member 4 and a first coupling port 9a is formed in the first hollow hub 9. The inlet port is formed in a male socket 4a which is connectable to a mating female socket of the external adapter 3 to allow passage of liquid from a distal liquid container (not shown) to, and through, the external adapter 3 to the patient's vein. An outlet port 16 of the valve assembly 1 is arranged in the second hollow hub 13 of the second valve member. When the valve assembly 1 is placed in its engaged or attached state, the valve assembly 1 allows for liquid passage between the inlet port (not shown) and the outlet port 16. The skilled person will understand that the terms “outlet port” and “inlet port” are interchangeable and only used for illustrative purposes since the flow of liquid may run from the inlet port towards the outlet port 16 or vice versa dependent of the type of application and delivery needs of a patient.

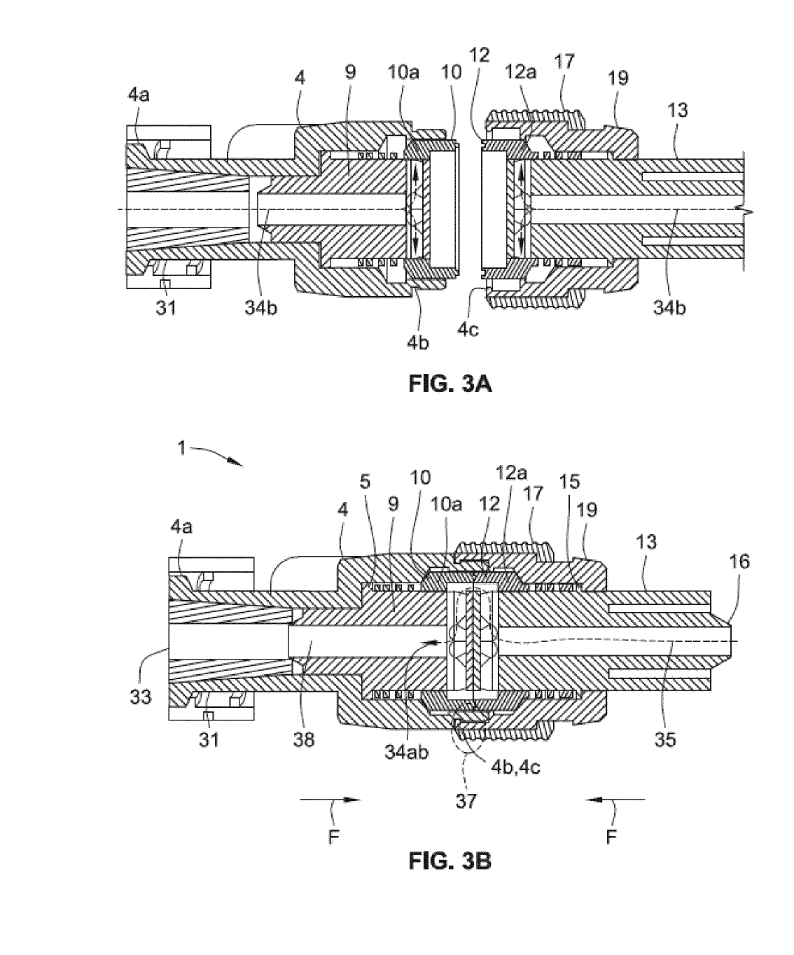

An annular slider 17 comprises a cylindrical inner surface contour mating to an outer surface contour of the first cylindrical housing member 4 and an outer surface contour of the second cylindrical housing member 19. The first cylindrical housing member 4 comprises a first locking member in form of a circumferential groove 4b (refer to FIG. 3A) engraved in the housing member 4. The second cylindrical housing member 19 comprises a second locking member in form of a mating circumferential ridge or collar 4c arranged on the housing member 19. The annular slider 17 functions as a third locking member which cooperates with the circumferential groove 4b and the circumferential ridge or collar 4c to form a locking mechanism having a well-defined, and preferably adjustable, detachment force or retention force. The annular slider 17 preferably comprises a corrugated outer surface allowing a medical professional to grasp and twist or rotate the annular slider 17 to adjust a detachment force of the valve assembly 1 as explained in further detail below.

A first controllable valve comprises cooperating features of the first hollow hub 9 and the first axially displaceable hollow member 10 that are arranged co-axially about a longitudinal axis 2 of the valve assembly 1. A circumferential end section of the first hollow hub 9 comprises a plurality of circumferentially disposed and sideward facing openings 9a that form the first outlet port. The plurality of sideward facing openings 9a are selectively blocked or left open in response to axial movement or displacement of the first hollow hub 9 relative to the first hollow member 10. The first hollow member 10 is able to move in sliding engagement with an outer contour of the first hollow hub 9 between a proximal position where the plurality of sideward facing openings 9a are open and a distal position where the respective pluralities of sideward facing openings are blocked by the first hollow member 10. In its distal position, a solid annular section 10a of the first axially displaceable hollow member 10 tightly surrounds the plurality of sideward facing openings 9a to block liquid flow through these. However, in the proximal position of the first axially displaceable hollow member 10, the solid annular section 10a is displaced towards a circumferential collar or ridge 5 such that the plurality of sideward facing openings 9a is left open or unblocked to enable liquid flow there through. The first axially displaceable hollow member 10 comprises a helical spring structure 10b fastened to the above-mentioned solid annular section 10a. The distal position of the first hollow member 10 is set or defined by the abutment or engagement of the circumferential collar 5 to the helical spring structure 10b when the latter is uncompressed or in a relaxed state. The proximate position of the first hollow member 10 is on the other hand set or defined by the abutment of the circumferential collar 5 to the helical spring structure 10b when the latter is in a fully or at least partly compressed or loaded state or condition such that the entire first hollow member 10 is displaced towards the circumferential collar 5 in the manner described above.

A second controllable valve comprises cooperating features of the second hollow hub 13 and the second axially displaceable hollow member 12 or second hollow member 12 in a manner similar to the one described above in connection with the first controllable valve. The second hollow hub 13 and the second hollow member 12 are arranged co-axially about the longitudinal axis 2. A circumferential end section of the second hollow hub 13 comprises a plurality of circumferentially disposed and sideward facing openings 13a that form a second coupling port providing a liquid interface or passage to the first coupling port 9a of the first valve member as explained in additional detail below. An outlet port 16 is arranged in an opposite end of the second hollow hub 13 to allow in-going or outgoing flow of liquid to/from the valve assembly 1. The plurality of sideward facing openings 13a are selectively blocked or left open in response to axial movement or displacement of the second hollow hub 13. The second hollow member 12 is able to move in sliding engagement with an outer contour of the second hollow hub 13 between a proximal position where the plurality of sideward facing openings 13a are open and a distal position where the respective pluralities of sideward facing openings are blocked by the second hollow member 10. In its distal position, a solid annular section 12a of the second axially displaceable hollow member 12 tightly surrounds or covers the plurality of sideward facing openings 13a to block liquid flow through these as illustrated in FIG. 3A). However, in the proximal position of the second axially displaceable hollow member 12, the solid annular section 12a is displaced towards a circumferential collar or ridge 5 such that the plurality of sideward facing openings 13a is left open or unblocked beneath the second hollow member 12 to enable liquid flow there through as illustrated in FIG. 3B) where the dotted arrow 34a,b indicates a through-going liquid flow path of the valve assembly. The second hollow member 12 comprises a helical spring structure 12b fastened to the above-mentioned solid annular section 12a. Both the first hollow member 10 and the second hollow member 12 are preferably formed as respective moulded thermoplastic elements such that each member is an integral element comprising both the spring structures 10b, 12b and the solid structures 10a, 12a.

The distal position of the second hollow member 12 is set or defined by the abutment or engagement of the circumferential collar 15 to the helical spring structure 12b when the latter is uncompressed or in a relaxed state. The proximate position of the second hollow member 12 is on the other hand set or defined by the abutment of the circumferential collar 15 to the helical spring structure 12b when the latter is in a fully or at least partly compressed or loaded state or condition such that the entire second hollow member 12 is displaced towards the circumferential collar 15 in the manner described above and explained in further detail in connection with FIG. 3 below.

When the valve assembly 1 is brought into its engaged or attached coupling state where the first and second cylindrical housing members 4,19, respectively, are brought into locked engagement by the operation of the above-described first and second locking members 4b, 4c, respectively, (refer to FIG. 3A) the first hollow member 10 is displaced towards the circumferential collar or ridge 5 and the second hollow member 12 is displaced towards the circumferential collar or ridge 15. The distal end surfaces of the first and second hollow hubs 9, 13 are brought into abutment such that the respective plurality of sideward facing openings 9a, 13a are placed adjacently to each other to allow passage of liquid between the first and second valve members as explained in additional detail below. Finally, the annular slider 17 is slid into co-axial engagement with the second cylindrical housing member 19 pushing the latter toward the first cylindrical housing member 4 to engage and lock these to each other.

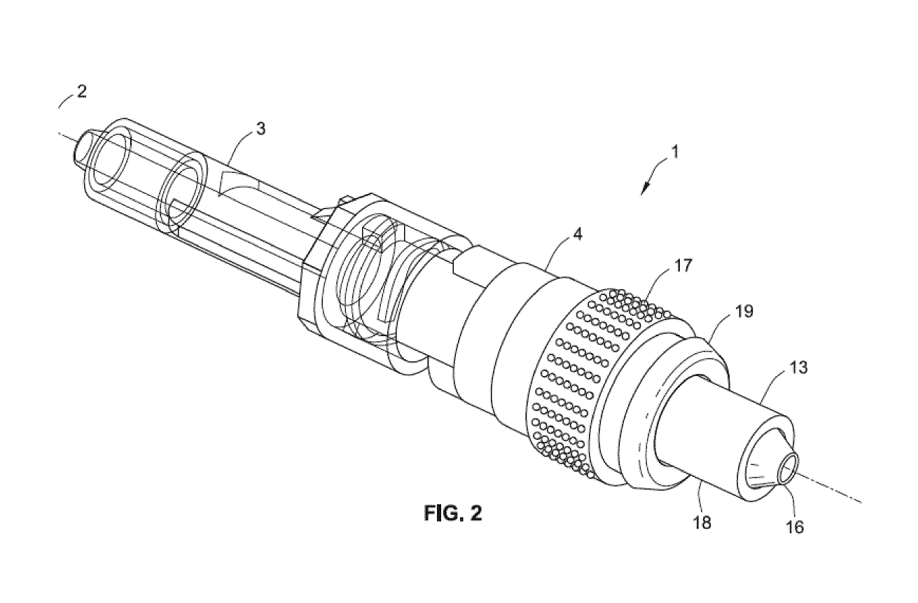

FIG. 2 is a perspective view of the valve assembly 1 disclosed on FIG. 1 in an engaged or attached state where the features discussed in connection with FIG. 1 have the same reference numerals. The adapter 3 does form part of the present valve assembly 1 but illustrates one exemplary type of medical tubing device that is connectable to the inlet port (not shown) of the valve assembly 1. In the illustrated engaged state, the valve assembly 1 allows for passage of liquid between the outlet port 16 and the inlet port (not shown) in opposite end of the assembly as discussed above.

The second hollow hub 13, which comprises the outlet port 16, further comprises an annular coupling section 18 functioning as a plug which can be press-fitted into an inner lumen of a medical delivery tube of appropriate diameter to create a substantially sealed connection thereto for passage of liquids.

FIGS. 3A) and 3B) are longitudinal cross-sectional views of the valve assembly 1 discussed above in connection with FIG. 1 and FIG. 2. FIG. 3A shows the valve assembly 1 in a disengaged or detached coupling state while FIG. 3B shows the valve assembly 1 in an engaged or attached coupling state for passage or through-going flow of liquid.

In FIG. 3A), the first hollow member 10 is arranged in its distal position such that the solid annular section or ridge 10a thereof surrounds and tightly engages or covers the plurality of sideward facing openings 9a to block liquid flow through these. Liquid may flow through an axially extending interior lumen 38 of the first hollow hub 9 as indicated by the dotted arrow but further flow is blocked at the plurality of sideward facing openings 9a by the solid annular ridge 10a. Thus, leakage of liquid from the first valve member at the first coupling port 9a is prevented in the detached state of the valve assembly 1. In the second valve member, the second hollow member 12 cooperates with the second hollow hub 13 in a corresponding manner to block liquid flow out of the plurality of sideward facing openings 13a.

In the assembled or attached state of the valve assembly 1 illustrated on FIG. 3B, the first hollow member 10 and the second hollow member 12 are arranged in their respective proximate positions where a pair of annular end surfaces are abutted to each other. Thereby, the solid annular section or ridge 10a of the first hollow member 10 has been slidingly displaced in proximal direction, i.e. towards the annular collar 5, so as to render the plurality of sideward facing openings 9a exposed or open below the solid annular section 10a of the first hollow member 10 with a larger inner diameter than an outer diameter of the first hollow hub 9. Likewise, the solid annular section 12a of the second hollow member 12 has also been slidingly displaced in proximal direction, i.e. towards the annular collar 15, so as to expose or open the plurality of sideward facing openings 13a below the solid annular section 12a of the second hollow member 12 with a larger inner diameter than an outer diameter of the second hollow hub 13. Consequently, liquid is allowed to flow through the inner lumen 35 of the second hollow hub 13 and upwards and downwards out of the plurality of sideward facing openings 13a, through a cylindrical volume below the abutted solid annular sections 10a, 12a and into the plurality of sideward facing openings 9a of the first hollow hub as schematically illustrated by the liquid flow path arrow 34ab of FIG. 3B). The liquid flow projects through an inner lumen 38 of the first hollow hub 9 to the outlet port 33 situated at the male socket 4a located at the end of the first valve member.

If the valve assembly 1, when placed in the attached coupling state illustrated in FIG. 3B), is subjected to a force along the longitudinal axis 2 (Refer to FIG. 2) in opposite direction to the indicated force vectors F, the locked engagement between the annular slider 17 and the first and second cylindrical housing members 4,19, respectively, is broken at a well-defined retention force, or detachment force, set by material properties of the annular slider 17, and respective dimensions of locking members 4b, 4c arranged on the first cylindrical housing member 4 and the second cylindrical housing member 19, respectively. The first locking members 4b comprise an annular and circumferential groove structure and the second locking member 4c comprises a mating annular and circumferential ridge structure. The annular slider 17 is displaceable along the longitudinal axis 2 and functions as third locking member surrounding and directly engaging the second locking member 4c and indirectly engaging the first locking member 4b. An interior cylindrical surface of the annular slider 17 preferably comprises a thread which mates to a corresponding thread arranged on an outer surface of the second cylindrical housing members 19 such that rotation of the annular slider 17 leads to axial displacement thereof.

The detachment or retention force required to switch the first and second valve members from the attached coupling state to the detached coupling state is also controlled by a distance or degree of overlap in axial direction between the first and second cylindrical housing members 4,19, respectively, and the annular slider 17 (i.e. overlap along the longitudinal axis 2 depicted on FIG. 2). The axial overlap distance of the illustrated embodiment of the valve assembly is indicated inside elliptical window 37. In the depicted position of the annular slider 17, the slider 17 completely surrounds an annular end portion of the first locking member 4 and an annular end portion of the second locking member 19 to provide a relatively large detachment force for example about 20 N. The detachment force is caused by the restraining of the annular end portions of the first and second locking members in radially outwardly direction caused by the inwardly directed retention force of the annular slider 17. Hence, a relatively large force is required to dismantle the first and second locking members from each other. However, by rotating the annular slider 17, the detachment force may be varied, in this case reduced, by rotating and thereby axially displacing the annular slider 17 towards the outlet port 16 such that the axial overlap distance between the annular slider 17 and the first and second locking members 4b, 4c is reduced. The reduced axial overlap distance allows the annular end portions of the first and second cylindrical housing members 4, 19 to dislodge at a smaller detachment force.

The variable detachment force is an advantageous feature because it allows the doctor or nurse to adapt the detachment force to particular patient's situation and/or to the type of vein catheter and/or strength of the fastening mechanism applied to the vein catheter.

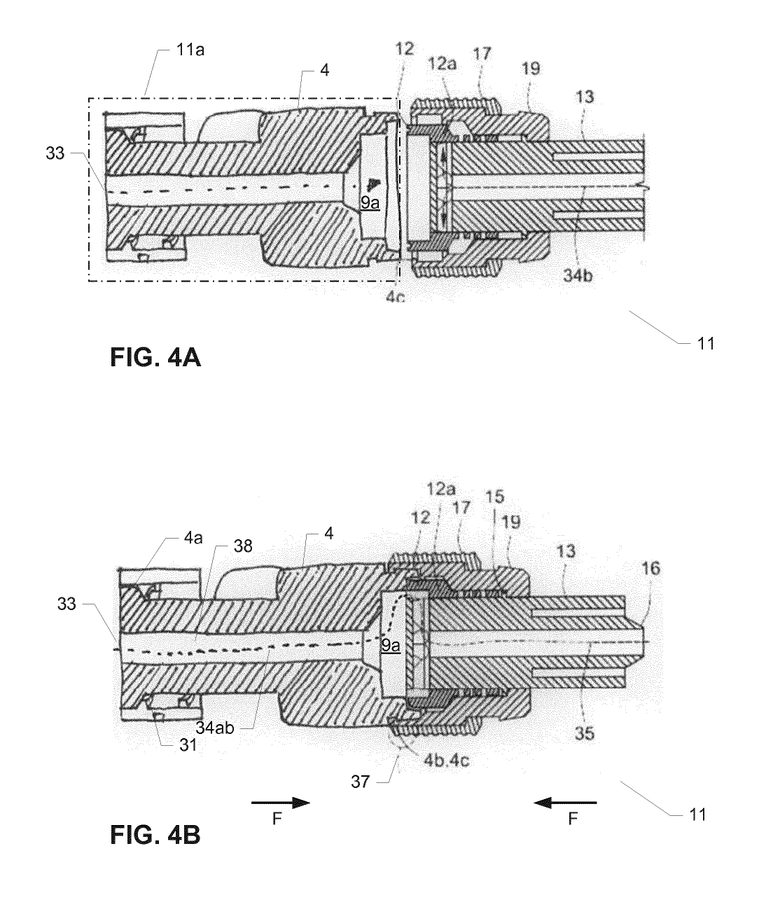

FIGS. 4A) and 4B) are longitudinal cross-sectional views of a valve assembly 1 in accordance with a second embodiment of the invention that only comprises a single controllable valve. FIG. 4A shows the valve assembly 11 in a disengaged or detached coupling state while FIG. 4B shows the valve assembly 11 in an engaged or attached coupling state for through-going flow of liquid. The present embodiment 11 of the valve assembly comprises a coupling member 11a which replaces the first valve member of the first embodiment of the invention described above. The first valve member is preferably identical to the second valve member of the first embodiment of the invention. The present embodiment of the valve assembly 11 accordingly shares numerous features with the first embodiment of the valve assembly 1 described above and similar features have been provided with identical reference numerals to assist comparison.

In FIG. 4A), the coupling member 11 a comprises a first cylindrical housing member 4. The first cylindrical housing member comprises an outlet port or opening 33 connectable via a male socket 4a to an inlet or port of a delivery tube or catheter which comprises mating engagement means. An opposite side of the first cylindrical housing member 4 comprises another or second coupling port 9a. A first locking member 4b is arranged on the first cylindrical housing member 4. The first locking members 4b comprise an annular and circumferential groove structure arranged proximate to the second coupling port 9a. A second cylindrical housing member 19 of a first controllable valve comprises a second locking member 4c with a mating annular and circumferential ridge structure. The coupling member 11a and the first valve member can accordingly transit from the engaged coupling state to the detached or disengaged coupling state or vice versa in the same manner as described above in connection with the first embodiment of the invention. The present valve assembly 11 furthermore comprises an annular slider 17 that is displaceable along a longitudinal axis of the valve assembly and functions similar to the previously described third locking member.

In the detached coupling state of the valve assembly depicted in FIG. 4A, a first hollow member 12 is arranged in its distal position such that a solid annular section or ridge 12a thereof surrounds and tightly engages or covers the plurality of sideward facing openings 13a (refer to FIG. 1) blocking liquid flow through these. Liquid may flow through an axially extending flow path 34b of a first hollow hub 13 as indicated by the dotted arrow 34b but further flow is blocked at the plurality of sideward facing openings 13a by the solid annular ridge 12a. Thus, leakage of liquid from the first valve member at the first coupling port 13a is prevented in the detached state of the valve assembly 11 in a corresponding manner to the liquid flow blocking function described above in the first valve assembly embodiment 1. However, in the coupling member 11a, the liquid can flow between the an outlet port 33 and the oppositely arranged coupling port 9a in the detached coupling state due to the lack of a controllable valve mechanism in the coupling member 11a.

In the engaged or attached state of the valve assembly 11 illustrated on FIG. 4B, the first hollow member 12 has been displaced to its proximate position by engagement with an annular end surface of the first cylindrical housing member 4 facing a mating annular end surface of the solid annular section or ridge 12a. Thereby, the solid annular ridge 12a of the first hollow member 12 has been slidingly displaced in proximal direction, i.e. towards an annular collar 15 (refer to FIG. 1), so as to render the plurality of sideward facing openings 13a exposed or open below a solid annular section 12a of the first hollow member 12 with a larger inner diameter than an outer diameter of the first hollow hub 13. Consequently, liquid is allowed to flow through the inner lumen 35 of the first hollow hub 13 and upwards and downwards out of the plurality of sideward facing openings 13a, constituting the first coupling port of the first valve, and into a cylindrical volume enclosing the coupling port 9a. The resulting liquid flow path formed in the engaged state of the valve assembly 11 is schematically illustrated by the liquid flow path arrow 34ab of FIG. 4B).

The user adjustable detachment force required to switch the coupling member and first valve member from the engaged coupling state to the detached coupling state is controlled as described above in connection with the first embodiment of the valve assembly 1.

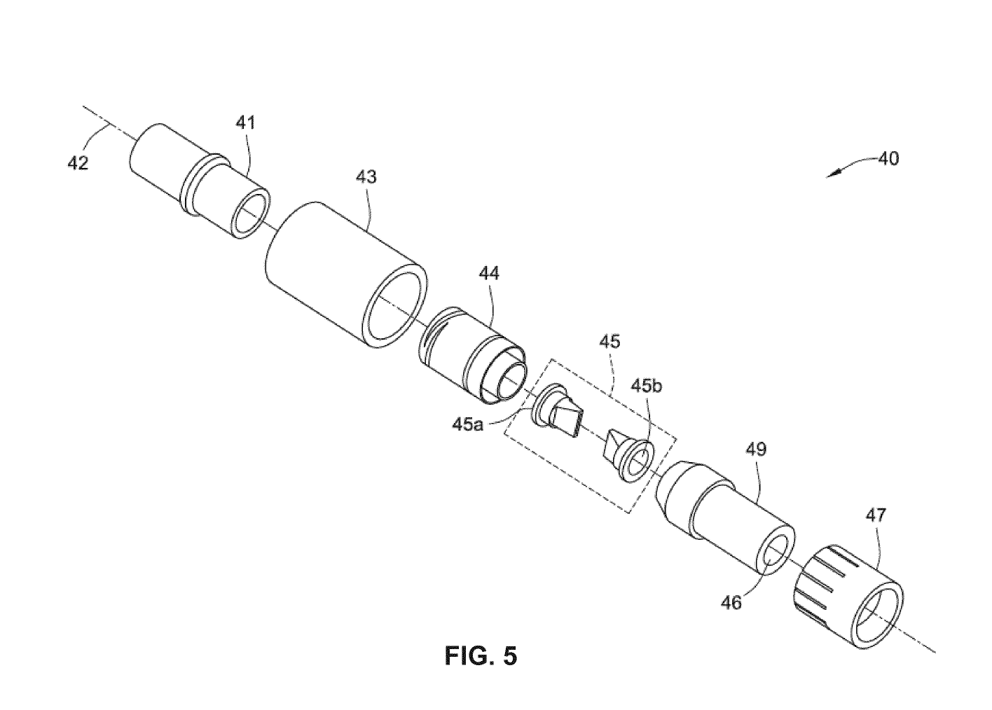

FIG. 5 is an exploded perspective view a valve assembly 40 in accordance with a second embodiment of the invention. The valve assembly 40 comprises a first valve member or assembly comprising a first hollow hub 41, a tubular sliding element 43, a first valve retaining element 44 and a first controllable valve comprising a half-portion of shared valve element 45. The valve assembly 40 comprises further a second valve member or assembly comprising a second valve retaining element 49, a second controllable valve comprising the residual or second half-portion of the shared valve element 45 and a tubular locking element 47. An inlet port (not shown) is formed in a distal end of the first hollow hub 41 having a cylindrical outer surface for press-fit coupling to a hollow delivery tube of mating shape. An outlet port 46 of the valve assembly 1 is arranged in an end section of the second valve retaining element 49. When the valve assembly 40 is placed in its engaged or attached coupling state, the assembly 40 allows liquid passage between the inlet port (not shown) and the outlet port 46. The skilled person will understand that the terms “outlet port” and “inlet port” are interchangeable and only used for illustrative purposes since the flow of liquid may run either from the inlet port towards the outlet port 16 or vice versa dependent of the type of application and delivery needs of a patient.

In an assembled state, the first half-portion of the shared valve element 45 is enclosed within a cone shaped interior volume of the first valve retaining element 44 mating to a cone shaped outer contour of the first half-portion of the shared valve element 45. Likewise, the second half-portion of the shared valve element 45 is enclosed within a cone shaped interior volume of the second valve retaining element 49 mating to a cone shaped outer contour of the second half-portion of the shared valve element 45. This is illustrated in further detail below. The first controllable valve is formed in the first half-portion of the shared valve element 45 which comprises a compressible elastomeric material such as rubber or silicone etc. A first coupling port (not shown) of the valve assembly 40 is formed at a central vertical section of the shared valve element 45 where the first half-portion and the second half-portion are integrally formed so as to provide single shared valve element 45 comprising both the first and second controllable valves. A liquid port 45a of the first half-portion of the shared valve element 45 leads to the first controllable valve and a liquid port 45b of the second half-portion leads to the second controllable valve. The operation of the first and second controllable valves is controlled by a valve control mechanism that is configured to compress the first half-portion and the second half-portion of the shared elastomeric valve element 45 in a direction perpendicular to the central longitudinal axis 42 to open the first and second valves for liquid flow as explained in further detail below.

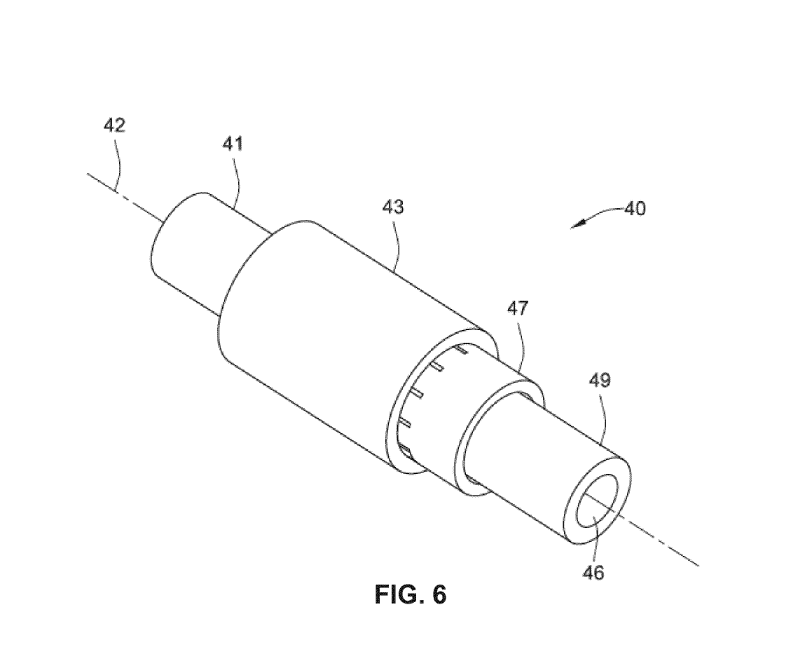

FIG. 6 is a perspective view of the valve assembly 40 in an assembled and engaged or attached state in accordance with the second embodiment of the invention. Liquid is allowed to flow through the valve assembly 40 in this coupling state from the inlet port (not shown) at the distal end of the first hollow hub 41 to the outlet port 46 or vice versa. A section of the tubular locking element 47 protrudes in longitudinal direction from the tubular sliding element 43 so as to allow a medical professional to grasp and twist this section of the tubular locking element 47 to adjust the detachment force of the detachable vent assembly 40.

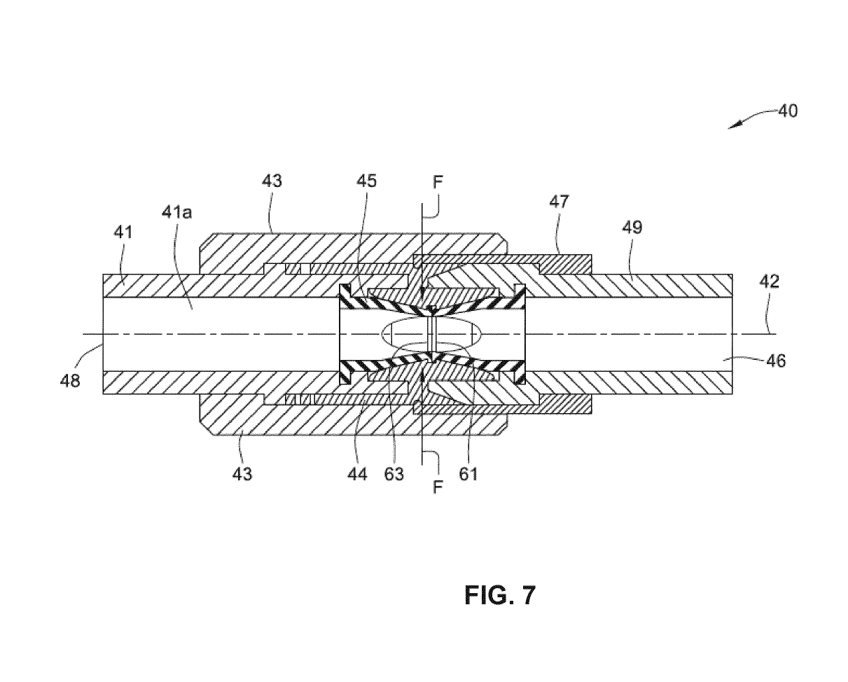

FIG. 7 is a central longitudinal cross-sectional view of the valve assembly 40 in accordance with the second embodiment of the invention depicted in the engaged or attached coupling state. Flow of liquid through the valve assembly 40 from the inlet port 48 to the outlet port 46 or vice versa is enabled as previously described. A controllable valve section is provided in a central portion of the shared elastomeric valve element 45. A first coupling port comprises a first membrane 63 having a substantially straight slit formed therein and the second coupling port is facing the first coupling port at an adjacent position and likewise comprises a second membrane 61 having a substantially straight slit formed therein. A valve control mechanism comprises the first valve retaining element 44 which surrounds the central portion of the shared elastomeric valve element 45 which comprises the first and second adjacently arranged coupling ports. Each of the first and second coupling ports changes from a collapsed state to an expanded state when the first valve retaining element 44 applies a transversally and inwardly directed force, as indicated by depicted force vectors “F”, to the shared elastomeric valve element 45 and to the first and second membranes 63, 61 arranged in the central portion of the shared elastomeric valve element 45. The shape of the first coupling port changes from the substantially straight slit in the first membrane 63, blocking liquid flow through the first membrane, to an approximately elliptical aperture, enabling liquid flow through the first membrane 63. A corresponding change of shape is induced to the second coupling port by the inwardly directed force from first valve retaining element 44. Thereby, the first and second coupling ports are simultaneously opened in response to the transversally and inwardly directed force applied by the first valve retaining element 44 as illustrated in further detail below to enable liquid flow through the valve assembly 40.

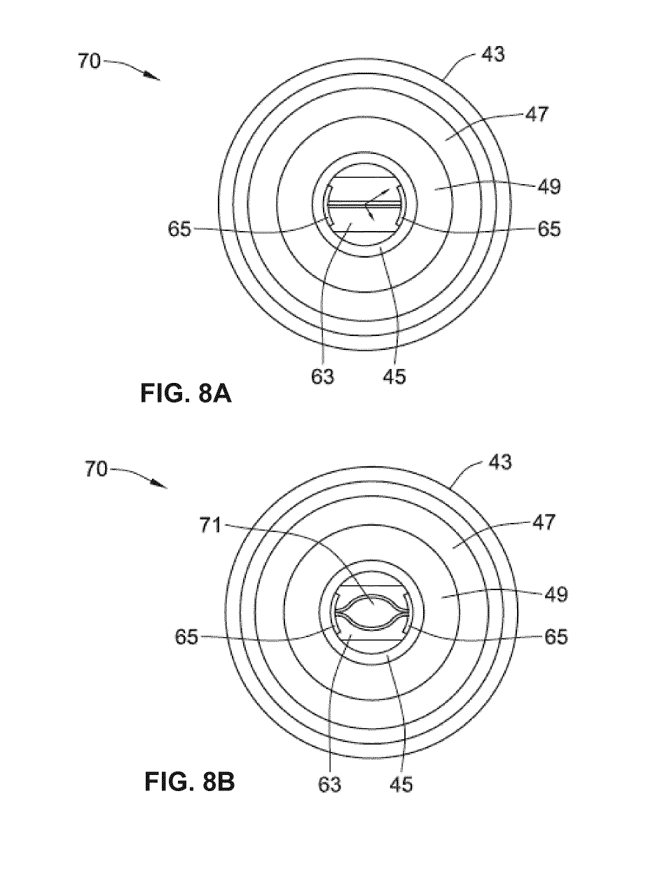

FIG. 8A is a transverse cross-sectional view of the valve assembly 40 in a disengaged or detached coupling state. The illustrated transverse view 70 is made at the central portion of the shared elastomeric valve element 45 which comprises the first and second adjacently positioned coupling ports. The tubular sliding element 43 is at the outmost periphery of the cross-sectional view partly enclosing and abutting the tubular locking element 47. The first valve retaining element 44 surrounds an outer peripheral surface of the shared elastomeric valve element 45 and comprises a pair of semi-cylindrical arms 65 engaging the first membrane 63 in which the first expansible coupling port is located. The straight slit in the first membrane 63 is closed in the illustrated detached coupling state of the valve assembly because the pair of semi-cylindrical arms 65 does not apply any significant horizontally directed force to the first membrane 63 in this coupling state. This blocking of the liquid passage through the first valve member prevents flow of liquid such as blood or medicine out of the first coupling port when the valve assembly 40 in placed in the disengaged coupling state. A corresponding closure of the second coupling port is induced as a result of the inwardly directed force from first valve retaining element 44. Thereby, the first and second coupling ports are simultaneously closed or blocked to block liquid flow through the valve assembly 40 and additionally block liquid flow through each separate valve member in the detached coupling state.

FIG. 8B is a transverse cross-sectional view of the valve assembly 40 made at the same location as FIG. 8A but this time in the engaged or attached coupling state of the valve assembly. In the attached coupling state the pair of semi-cylindrical arms 65 project further inwardly in horizontal direction compared to the detached coupling state illustrated in FIG. 8A. The pair of semi-cylindrical arms 65 therefore both engages and applies a predetermined force to the first membrane 63 so as to expand the straight slit into an essentially elliptical opening 71 in the first membrane 63 and enable liquid flow there through. A corresponding change of shape is induced to the second coupling port arranged in the second membrane 61 by the pair of semi-cylindrical arms 65 so as to simultaneously open the first and second coupling ports and enable liquid flow through the valve assembly 40.

1. A valve assembly for insertion in a liquid flow path between a drain or catheter and a liquid vessel or container, the valve assembly comprising:
a first valve member comprising:
an inlet port connectable to a first delivery tube and arranged at a first side of the first valve member;
a first coupling port arranged at a second side of the first valve member;
a first locking member;
a first controllable valve adapted to selectively block or enable liquid flow between the inlet port and the first coupling port in accordance with a valve control mechanism; and
a coupling member comprising:
an outlet port connectable to a second delivery tube and arranged at a first side of the coupling member;
a second coupling port arranged at a second side of the coupling member;
a second locking member configured for mating engagement with the first locking member on the first valve member;
wherein the valve control mechanism is responsive to a coupling state of the first and second locking members.

2. A valve assembly according to claim 1, wherein the coupling member comprises:
a second controllable valve for selectively blocking or enabling liquid flow between the outlet port and the second coupling port of the coupling member in accordance with the valve control mechanism.

3. A valve assembly according to claim 1, wherein the first and second locking members are configured to transit from an engaged coupling state to a detached coupling state at a detachment force between 0.5 N and 20 N, measured in a longitudinal axial direction of the valve assembly.

4. A valve assembly according to claim 1, wherein the first and second locking members comprise a mating annular ridge and groove structure.

5. A valve assembly according to claim 1, comprising an axially displaceable third locking member surrounding and engaging at least one of the first and second locking members to provide a user adjustable detachment or retention force.

6. A valve assembly according to claim 5, wherein third locking member is rotatably mounted about one of the first and second locking members.

7. A valve assembly according to claim 6, wherein an interior surface of the third locking member and an outer surface of the first or the second locking member comprises mating threads.

8. A valve assembly according to claim 6, wherein the third locking member comprises a corrugated outer surface.

9. A valve assembly according to claim 2, wherein
the first controllable valve comprises a first hollow hub having formed therein a plurality of sideward facing openings and a first axially displaceable hollow member surrounding at least a portion of the first hollow hub to selectively open or block the plurality of sideward facing openings; and
the second controllable valve comprises a second hollow hub having formed therein a plurality of sideward facing openings and a second axially displaceable hollow member surrounding at least a portion the second hollow hub to selectively open or block the plurality of sideward facing openings.

10. A valve assembly according to claim 9, wherein the first and second valve members are configured to move each of the first and second axially displaceable hollow members between a proximal position where the respective pluralities of sideward facing openings are open and a distal position where the respective pluralities of sideward facing openings are blocked by the first and second axially displaceable hollow members.

11. A valve assembly according to claim 9, wherein:
the first axially displaceable hollow member comprises a first spring configured to engage a circumferential collar on the first hollow hub; and
the second axially displaceable hollow member comprises a second spring configured to engage a circumferential collar on the second hollow hub.

12. A valve assembly according to claim 8, wherein:
the first hollow hub and the first axially displaceable hollow member are arranged in sliding engagement and co-axially about a longitudinal axis of the valve assembly; and
the second hollow hub and the second axially displaceable hollow member are arranged in sliding engagement and co-axially about the longitudinal axis of the valve assembly.

13. A valve assembly according to claim 11, wherein:
the first valve member comprises a first hollow housing at least partly surrounding the first hollow hub and the second valve member comprises a second hollow housing at least partly surrounding the second hollow hub; and
the first and second hollow housings are configured to engage and axially displace the first and second hollow hubs, respectively, to axially compress the first and second springs against their respective circumferential collars.

14. A valve assembly according to claim 1, wherein the first valve member and the coupling member are rotatable relative to each other about a longitudinal axial direction of the valve assembly in an engaged coupling state of the first and second locking members.

15. A valve assembly according to claim 2, wherein the first controllable valve comprises a first compressible elastomeric member having the first coupling port formed therein; and
the second controllable valve comprises a second compressible elastomeric member having the second coupling port formed therein;
wherein the valve control mechanism is configured to compress the first and second compressible elastomeric members to change shapes of the first and second coupling ports.
